(19) United States
(12) Patent Application Publication
(10) Pub. No.: US 2023/0226280 A1
(43) Pub. Date: Jul. 20, 2023

(54) SYRINGE INJECTOR WITH A FEEDBACK MECHANISM

(71) Applicant: OcuJect, LLC, Newport Beach, CA (US)

(72) Inventor: Leonid E. Lerner, Corona Del Mar, CA (US)

(21) Appl. No.: 18/000,925

(22) PCT Filed: Sep. 24, 2021
(86) PCT No.: PCT/US2021/071583

§ 371 Date: Dec. 6, 2022

Related U.S. Application Data
(60) Provisional application No. 63/083,717, filed on Sep. 25, 2020.

(51) Int. Cl. A61M 5/315

(52) U.S. Cl. CPC A61M 5/31513

(57) ABSTRACT

An injection device includes one or more of a barrel having an open proximal end, an open distal end, and a sidewall therebetween; a plunger; and a conduit or connector. The barrel may be configured to house a volume of therapeutic agent. The plunger may have a proximal end, a distal end, and an elongate body. The conduit or connector may be operably attached to the open distal end of the barrel. The distal end of the barrel can include a variable inner diameter segment configured such that movement of the plunger to the variable inner diameter segment of the distal end of the barrel creates an audible and/or tactile feedback sensation to a user.

### CROSS-REFERENCE TO RELATED APPLICATIONS

This application is a national stage application of International Patent Application No. PCT/US2021/071583, filed on Sep. 24, 2021, which claims priority to U.S. Provisional Pat. Application No. 63/083717, filed on Sep. 25, 2020. The disclosure of each of these prior applications is hereby incorporated by reference in its entirety.

### BACKGROUND

A wide variety of conventional injection devices can be utilized to inject one or more therapeutic agents. Injection devices that can include feedback features to indicate and/or facilitate completion of injection are needed and are disclosed herein.

### SUMMARY

In some embodiments, disclosed herein is an injection device, comprising one or more of: a barrel comprising an open proximal end, an open distal end, and a sidewall therebetween, the barrel configured to house a volume of therapeutic agent; a plunger comprising a proximal end, a distal end, and an elongate body; and a conduit or connector operably attached to the open distal end of the barrel. The distal end of the barrel can comprise a variable inner diameter segment configured such that movement of the plunger to the variable inner diameter segment of the distal end of the barrel creates an audible and/or tactile feedback sensation to a user.

In some embodiments, the device comprises a piston syringe.

In some embodiments, an inner diameter of the barrel proximal to the variable inner diameter segment is substantially constant.

In some embodiments, the variable inner diameter segment comprises a first segment and a second segment distal to the first segment, the second segment comprising an inner diameter that is greater than an inner diameter of the first segment.

In some embodiments, the inner diameter of the second segment is between about 0.1 mm and about 0.5 mm greater than the inner diameter of the first segment.

In some embodiments, the inner diameter of the second segment is about 0.3 mm greater than the inner diameter of the first segment.

In some embodiments, the second segment has an axial length of between about 0.05 mm and about 0.20 mm.

In some embodiments, the device also includes a transition zone between the first segment and the second segment.

In some embodiments, the transition zone comprises an axial length that is less than, and within about 0.05 mm of the length of the second segment.

In some embodiments, the transition zone comprises an axial length that is less than, and within about 0.03 mm of the length of the second segment.

In some embodiments, the transition zone is slanted at an angle Z with respect to a long axis of the sidewall of the barrel, wherein the angle Z is less than about 90 degrees.

In some embodiments, the transition zone is slanted at an angle Z with respect to a long axis of the sidewall of the barrel, wherein the angle Z is less than about 45 degrees.

In some embodiments, the transition zone is slanted at an angle Z with respect to a long axis of the sidewall of the barrel, wherein the angle Z is greater than about 45 degrees.

In some embodiments, the movement of the plunger to the variable inner diameter segment of the distal end of the barrel creates negative pressure within the barrel sufficient to fully inject the entire volume of the barrel.

In some embodiments, the distal end of the plunger comprises a variable inner diameter segment that is substantially the same length and inner diameter as the variable inner diameter segment of the distal end of the barrel.

In some embodiments, the variable inner diameter segment at the distal end of the barrel is less than about 10% of the total axial length of the barrel.

In some embodiments, the conduit or connector comprises a luer connector.

In some embodiments, the volume is between about 0.25 ml and about 20 ml.

In some embodiments, the volume is about 1 ml.

In some embodiments, disclosed herein is a method of injecting a therapeutic agent, comprising: providing an injector comprising a barrel comprising an open proximal end, an open distal end, and a sidewall therebetween, the barrel configured to house a volume of therapeutic agent; a plunger comprising a proximal end, a distal end, and an elongate body; and a conduit or connector operably attached to the open distal end of the barrel, wherein the distal end of the barrel comprises a variable inner diameter segment; and actuating the plunger to purge the volume of therapeutic agent out of the barrel, such that movement of the plunger to the variable inner diameter segment of the distal end of the barrel creates an audible and/or tactile feedback sensation to a user.

In some embodiments, movement of the plunger to the variable inner diameter segment of the distal end of the barrel creates negative pressure within the barrel to accentuate an audible and/or tactile feedback sensation to the user.

In some embodiments, the injector comprises a piston syringe.

In some embodiments, an inner diameter of the barrel proximal to the variable inner diameter segment is substantially constant.

In some embodiments, the variable inner diameter segment comprises a first segment and a second segment distal to the first segment, the second segment comprising an inner diameter that is greater than an inner diameter of the first segment.

In some embodiments, the inner diameter of the second segment is between about 0.1 mm and about 0.5 mm greater than the inner diameter of the first segment.

In some embodiments, the inner diameter of the second segment is about 0.3 mm greater than the inner diameter of the first segment.

In some embodiments, the second segment has an axial length of between about 0.05 mm and about 0.20 mm.

In some embodiments, the variable inner diameter segment also comprises a transition zone between the first segment and the second segment.

In some embodiments, the transition zone comprises an axial length that is less than, and within about 0.05 mm of the length of the second segment.

In some embodiments, the transition zone comprises an axial length that is less than, and within about 0.03 mm of the length of the second segment.

In some embodiments, the transition zone is slanted at an angle Z with respect to a long axis of the sidewall of the barrel, wherein the angle Z is less than about 90 degrees.

In some embodiments, the transition zone is slanted at an angle Z with respect to a long axis of the sidewall of the barrel, wherein the angle Z is less than about 45 degrees.

In some embodiments, the transition zone is slanted at an angle Z with respect to a long axis of the sidewall of the barrel, wherein the angle Z is greater than about 45 degrees.

In some embodiments, the therapeutic agent comprises an anti-VEGF agent.

In some embodiments, the anti-VEGF agent is selected from the group consisting of conbercept, ranibizumab, razumab, aflibercept, bevacizumab, and brolucizumab.

In some embodiments, the therapeutic agent comprises an immunomodulatory agent.

In some embodiments, the immunomodulatory agent comprises a steroid.

In some embodiments, the therapeutic agent is pre-filled within the barrel.

In some embodiments, disclosed herein is an injection device, comprising: a barrel comprising an open proximal end, an open distal end, and a sidewall therebetween, the barrel configured to house a volume of therapeutic agent and defining a therapeutic agent reservoir; a plunger comprising a proximal end, a distal end, and an elongate body; and a conduit or connector operably attached to the open distal end of the barrel, wherein the distal end of the barrel comprises a variable inner diameter segment configured such that movement of the plunger to the variable inner diameter segment of the distal end of the barrel creates an audible and/or tactile feedback sensation to a user while passing through the variable inner diameter segment, and wherein the variable inner diameter segment comprises a first, narrower proximal part, a second, wider distal part, and a third angled transition zone part between the first part and the second part.

In some embodiments, the conduit comprises a removable or non-removable conduit.

In some embodiments, the conduit comprises a needle.

In some embodiments, the connector comprises a universal luer connector.

In some embodiments, the second part is adjacent to a distal wall of the therapeutic agent reservoir.

In some embodiments, the distal end of the plunger is sized and configured to pass through the first part, second part, and third part of the variable inner diameter segment without losing an air and/or fluid tight seal.

In some embodiments, an injector device can comprise, consist essentially of, consist of, or not comprise any number of features of the disclosure.

In some embodiments, a method can comprise, consist essentially of, consist of, or not comprise any number of features of the disclosure.

### BRIEF DESCRIPTION OF THE DRAWINGS

FIG. 1 illustrates an embodiment of an injection device, such as a piston syringe, according to some embodiments of the invention.

FIGS. 2 and 3 illustrate a portion of a distal end region of an injector 1, such as the injection device or a syringe as illustrated and described in FIG. 1.

FIG. 4 schematically illustrates the seal portion at or proximate the distal end of the plunger of the injector of FIG. 1, according to some embodiments.

FIG. 5 illustrates one embodiment of an injection device, such as a piston syringe, that includes plunger with two seals and a plunger sealing cap.

FIG. 6A is a side view of the injection device of FIG. 5.

FIG. 6B is a cross-section view of the injection device of FIG. 6A taken along line 6B-6B.

FIG. 6C is an exploded view of the multi-segment piston of the injection device of FIGS. 6A and 6B.

FIG. 6D is an exploded detailed view of the distal segment and the distal end of the proximal segment of the multi-segment piston of FIG. 6C taken along line 6D-6D.

### DETAILED DESCRIPTION

In some embodiments, an injector, such as syringe can include one, two, or more feedback features that indicates to a user that injection is complete. This feature, can include, for example, an audible and/or tactile feedback feature, such as a click, for example. Alternatively or in addition, the feature could be a visual feature. Feedback features can be very advantageous to indicate to a user that they can stop applying pressure to a control, such as a syringe plunger, for example, to reduce the risk of unintended migration of a needle that may be embedded in the patient’s anatomy that may result in tissue damage, for example, preventing the needle tip from migrating into an undesirable body structure or tissue plane with excessive force and/or time applied to syringe plunger that may be prolonged without such a feedback feature. Furthermore, feedback features can save a user time by knowing exactly when injection is complete. Importantly, in some embodiments feedback features may prevent an incomplete dose delivery by indicating to the user when the entire dose has been injected. Moreover, the feedback features may actually facilitate or expedite completion of the expulsion of the volume of the therapeutic agent from the syringe barrel. This may shorten the injection procedure time and keep it more consistent between injections, thus reducing patient’s discomfort and/or potential risk for infection that may increase with a longer procedure time, for example. In some embodiments, the feedback feature is distinctly observed when the injector, e.g., syringe barrel, is filled with a media, such as a liquid, for example, a therapeutic agent such as a drug with a relatively high viscosity. In some embodiments, the feedback feature can also be observed when the syringe is empty, or filled only with air. In some embodiments, the audible and/or tactile feedback feature is present only at the end of injection, and activated by a plunger reaching the distal-most end of the barrel. In some embodiments, the feedback feature may be particularly useful to ensure the complete dose delivery for small-volume pharmaceuticals. For example, the total dose of 0.1 ml or less. In another example, the total dose is 0.05 ml, which is a standard dose for certain intravitreal therapeutic agents, for example. Some can include, for example, aflibercept, conbercept, ranibizumab, razumab, bevacizumab, brolucizumab, fluocinolone, triamcinolone, dexamethasone, verteporfin, pegaptanib, ocriplasmin, combination pharmaceuticals containing the aforementioned agents, and other agents, including but not limited to intravitreal or intraocular medicines injected into the eye.

In some embodiments, a reservoir can be configured to hold one, two, or more therapeutic agents. A therapeutic agent can be any agent or combination of agents suitable for delivery to a patient, such as, for example, a drug, a biosimilar, an antibody, an antigen, a growth factor, a protein, a vector (e.g., a viral or plasmid vector), and the like. The reservoir could be, for example, a piston syringe with a variable (or uneven internal diameter barrel), such as at the distal tip of the barrel. In some embodiments, the drug reservoir can also include a fixed (or even) outer external diameter barrel. In some embodiments, the sidewall of the barrel is continuous from the proximal opening to the distal opening, without any additional openings. The drug reservoir can include a plunger configured to move in a direction to inject the therapeutic agent (e.g., drug) out of the reservoir.

In some embodiments, the proximal-most segment of the inner diameter of the barrel is constant (e.g., having a smooth constant or substantially constant diameter), and the distal-most 1%, 0.9%, 0.8%, 0.7%, 0.6%, 0.5%, 0.4%, 0.3%, 0.2%, 0.1%, or more or less of the axial length of the barrel is variable, or ranges including any two of the foregoing values.

For example, a drug reservoir such as a piston syringe can include a barrel with a first, narrower inner-diameter segment and a second, wider inner-diameter segment distal to the narrower inner-diameter segment, and in some cases separated by a transition zone. The device can also include a plunger.

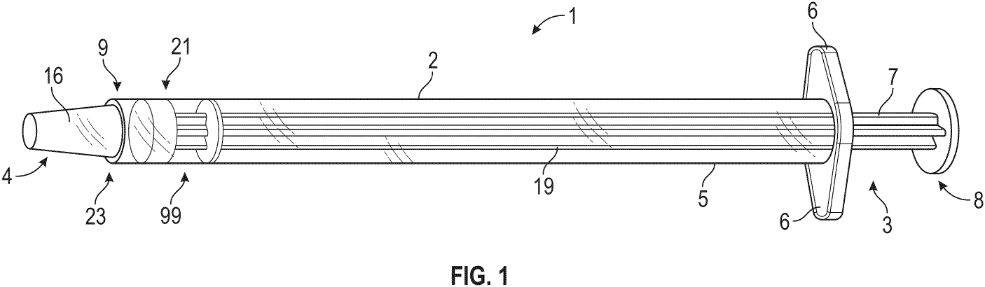

FIG. 1 illustrates an embodiment of an injection device, such as a piston syringe 1, according to some embodiments of the invention. The syringe 1 can include a barrel 2 including an open proximal end 3, an open distal end 4, and a sidewall 5 forming a chamber configured to receive a therapeutic agent, such as a drug for example. The open proximal end 3 can include radially extending wing portions 6 configured to serve as ergonomic grips in some embodiments. The distal end 4 of the syringe barrel 1 can include a conduit 16, or a connector for a drug conduit, such as a luer connector, needle injector, and/or an injector attachment with a slidable sleeve and other features as disclosed, for example, in U.S. Pat. No. 9,504,603 to Lerner, which is hereby incorporated by reference in its entirety. The distal end 4 of the barrel 2 can include one or more feedback features 99 that indicate to a user that injection is complete, as further discussed and illustrated herein.

Still referring to FIG. 1, the barrel 2 can include indicia of measurement, such as volume markers, for example, spaced evenly or unevenly apart along its length. The barrel 2 could have a transparent or translucent sidewall to allow visual inspection of the volume of the therapeutic agent in some embodiments. A plunger 7 can include a proximal end 8, distal end 9, and an elongate member 19 therebetween, and can be configured to travel axially within the sidewall 5 of the barrel 2. The distal end 9 of the plunger can include a stop surface 23 such as a radially outward extending ring configured to prevent the plunger 7 from proximally decoupling from the barrel 2 of the syringe 1. The distal end 9 of the plunger 7 can also include a seal 21, such as at or near its distal end 9 that can be closest to, or touch a drug conduit 16 or connector to a drug conduit, such as a luer or other connector, for example.

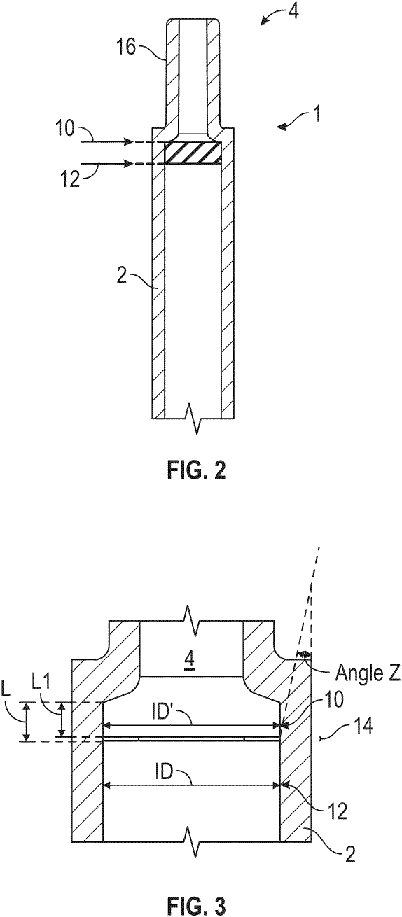

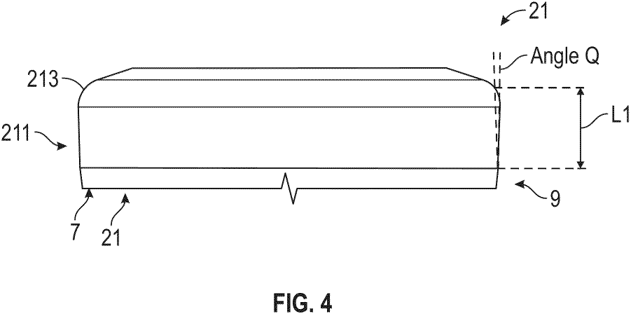

FIG. 2 illustrates a portion of a distal end 4 of an injector 1, such as a syringe such as that illustrated and described in FIG. 1, with feedback features according to some embodiments, and shown in greater detail in FIG. 3. A wider inner-diameter segment 10 with inner diameter ID′ is located distally to the narrower segment 12 with inner diameter ID (and directly adjacent/connected to the narrower segment in some embodiments, or separated by a transition zone 14), and is closer to the drug conduit 16 or a connector for a drug conduit 16, such as, for example, a luer connector. In some embodiments, the distal end of the wider inner-diameter segment 10 defines the terminus of axial travel of a plunger 7 or other injection element. In other words, the wider segment 10 can be located at the distal end 4 of the barrel 2 towards the drug conduit 16 or connector to a drug conduit 16, which can be, for example, a luer connector.

In some embodiments, the ID′ is equal to, or substantially equal to the ID + 0.3 mm ± 0.1 mm, 0.2 mm, or 0.3 mm, or more or less.

Still referring to FIG. 3, in some embodiments, the wider segment 10 of the barrel of a drug reservoir has a length L including the length of a slanted transition zone 14, for example, about 0.1 mm +/- 0.03 mm long that is slanted at angle Z to the barrel’s long axis. In some embodiments, the transition zone 14 between the narrower (proximal) and wider (distal) segments 12, 10 may have a length of, for example, between 0.01 mm and 0.5 mm, between 0.015 mm and 0.25 mm, or about, at least about, or no more than about 0.01 mm, 0.015 mm, 0.02 mm, 0.03 mm, 0.04 mm, 0.05 mm, 0.075 mm, 0.10 mm, 0.15 mm, 0.20 mm, 0.25 mm, 0.30 mm, 0.35 mm, 0.40 mm, 0.45 mm, 0.50 mm, or more or less, or ranges including any two of the foregoing values.

In some embodiments, the transition zone between the narrower segment 12 and the wider segment 10 may be tapered, either gradually or abruptly. In one embodiment, the taper angle Z is shallow, for example, less than about 90 degrees. In another embodiment, the transition 14 between the wide and narrow segments 10, 12 is between about 1 degree and about 45 degrees, or between about 5 degrees and 30 degrees, or about, at least about, or no more than about 1, 5, 10, 15, 20, 25, 30, 35, 40, 45 degrees, or more or less, or ranges including any two of the foregoing values. In some embodiments, the taper angle Z can be greater in order to create a click effect, such as about or at least about 45, 50, 55, 60, 65, 70, 75, 80, 85, 90 degrees, or more, or ranges including any two of the foregoing values.

The length of the wider segment 10 without the transition zone 14 can be, for example, L1 = L - 0.1 mm ± 0.005 mm, 0.01 mm, 0.02 mm, 0.025 mm, 0.03 mm, 0.04 mm, 0.05 mm, or more or less, or other values or ranges as disclosed elsewhere herein. In some embodiments, the difference in dimension between the wider and narrower inner-diameter segments 10, 12 is between about 0.01 mm and about 1 mm. In another embodiment, the size (dimension) difference between the wider and narrower inner-diameter segments 10, 12 is between about 0.015 mm and about 0.05 mm, between about 0.02 mm and about 0.2 mm, about, at least about, or no more than about 0.005 mm, 0.01 mm, 0.02 mm, 0.03 mm, 0.04 mm, 0.05 mm, 0.06 mm, 0.07 mm, 0.08 mm, 0.09 mm, 0.10 mm, 0.11 mm, 0.12 mm, 0.13 mm, 0.14 mm, 0.15 mm, 0.2 mm, 0.3 mm, 0.4 mm, 0.5 mm, 0.6 mm, 0.7 mm, 0.8 mm, 0.9 mm, 1.0 mm, or more or less, and ranges including any two of the foregoing values. In some embodiments, the size difference between the wider and narrower inner-diameter segments 10, 12 is between about 0.001% and about 1%, such as about, at least about, or no more than about 0.001%, 0.005%, 0.01%, 0.02%, 0.03%, 0.04%, 0.05%, 0.06%, 0.07%, 0.08%, 0.09%, 0.10%, 0.15%, 0.20%, 0.25%, 0.30%, 0.40%, 0.50%, 0.75%, 1%, or more or less, or ranges including any two of the foregoing values.

In some embodiments, at least one or more parts within the plunger seal/stopper 21 can be sized, shaped, and otherwise configured to produce an audible and/or tactile click or other feedback while passing through the transition zone 14 into the wider inner-diameter distal segment 10 while the plunger seal/stopper 21 maintains the air- and water-tight seal with the barrel’s 2 inner diameter throughout the variable inner diameter segment.

In one embodiment, the drug reservoir, for example a 1 ml piston syringe, has a 4.67 mm internal-diameter barrel 2 and a 4.71 mm larger diameter segment 10 at the distal end 4 of the barrel 2 adjacent to the drug conduit 16 or a connector to a drug conduit 16, for example a luer connector.

In some embodiments, the drug reservoir can be sized and configured to have a volume of 0.23 ml, 0.3 ml, 0.5 ml, 1 ml, 2 ml, 2.5 ml, 3 ml, 4 ml, 5 ml, 10 ml, 15 ml, 20 ml, 25 ml, 30 ml, 40 ml, 50 ml, or more or less, or ranges including any two of the foregoing values.

In some embodiments, the axial length of the wider segment 10 is, for example, between about 0.1 mm and about 3 mm, between about 0.3 mm and about 2 mm, between about 0.5 mm and about 1.5 mm, or about 1 mm in length, about, at least about, or no more than about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5 mm or more or less, or ranges including any two of the foregoing values.

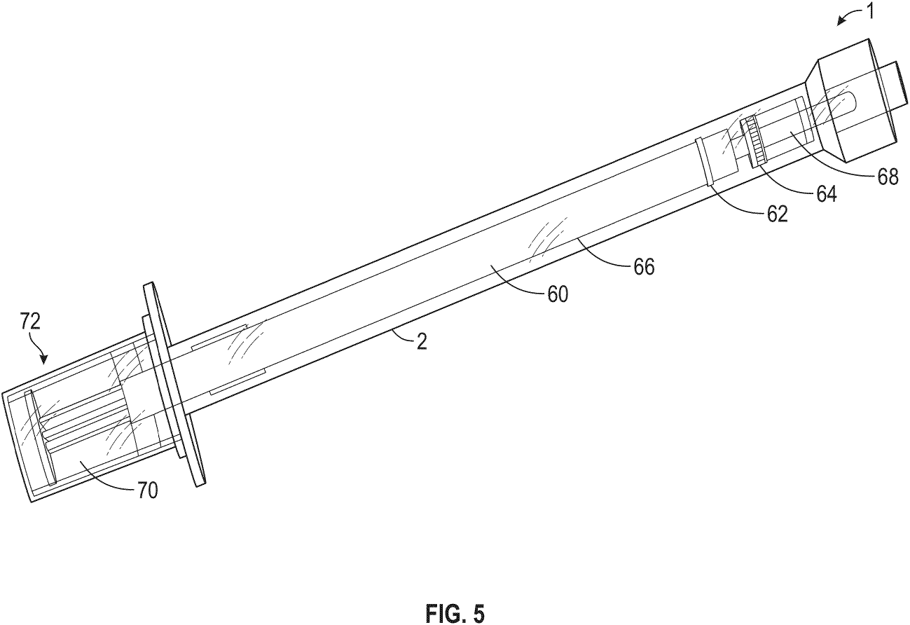

FIG. 4 schematically illustrates the seal portion 21 at or proximate the distal end 9 of the plunger 7, according to some embodiments. The plunger seal 21 at its distal tip can include a wider segment 211 that has a length approximately equal to L1. L1 can be equal to, for example, L - 0.1 mm +/- 0.01 mm, 0.02 mm, 0.03 mm, or more or less, or other values or ranges as disclosed elsewhere herein.

The length of the larger-ID wider segment 10 may vary but in some embodiments is proportionally similar to the length of the plunger seal or stopper 21 (L : L1).

The length of the transition zone 14 may vary, but in some embodiments is proportionally sized based on the size and/or volume of the syringe barrel 2 and the length of the plunger seal or stopper 21, or any component of the seal or stopper 21.

In one example, as shown in FIG. 4, the side walls of the plunger seal 21 are slanted at an angle Q relative to the longitudinal axis of the plunger 7. In one example, the angle (Q) is steep enough to create an audible and/or tactile click feature without impeding the plunger movement, for example, less than about 45 degrees. In another embodiment, the angle Q is between about 1 and about 25 degrees, or between 5 and 20 degrees. In some embodiments, the angle Q is between about 1 degree and about 25 degrees, or between about 5 degrees and 20 degrees, or about, at least about, or no more than about 1, 5, 10, 15, 20, 25, 30, 35, 40, 45 degrees, or more or less, or ranges including any two of the foregoing values. In some embodiments, the angle Q can be greater in order to create a click effect, such as about or at least about 45, 50, 55, 60, 65, 70, 75, 80, 85, 90 degrees, or more, or ranges including any two of the foregoing values. The side surface of plunger seal 211 can include a rounded distal edge 213 to facilitate a click feature without impeding the plunger movement through the transition zone in the barrel wall.

In some embodiments, the particular geometry and dimensions of the enlarged inner diameter distal segment 10 with an angled or slanted transition zone 14 and the plunger seal 21 that may have an angled, slated or rounded edge interfacing with the inner wall of the barrel 2 can ensure that the water-tight seal between the plunger’s 7 distal end 9 and the inner wall of the barrel 2 is maintained through the transition zone 14 and the click or feedback feature does not disrupt the water-tight seal.

The particular geometry and dimensions of some embodiments of injectors, such as, for example, including an enlarged inner diameter distal segment 10 with an angled or slanted transition zone 14 may create a negative pressure (e.g., suction effect/force that is directed distally and ensures the completion of injection) that enhances/facilitates the plunger 7 movement distally at the end of injection, which ensures the delivery of the entire dose of the medication. Thus, the negative pressure effect/force at the end of injection ensures consistent delivery of the complete medication dose, which can be particularly important for small-dose medicines, for example, those used in volumes of about 0.1 ml or less, or 0.05 ml or less. This is distinct from other mechanisms that can create an obstruction or positive pressure that impede the plunger 7 movement at the end of injection, which may cause incomplete injection. Indeed, syringes 1 such as those described herein may be configured to generate a negative pressure in conjunction with and/or resulting from the end-click, feedback feature, which facilitates complete fluid injections. For example, the variable inner diameter segment at the distal end of the barrel 2 creates a negative pressure or lower friction force compared to the rest of the barrel. Thus, the audible and/or tactile click at the end of injection is associated with a negative pressure or lower resistance point to the plunger advancement, e.g., at the end injection of a medication from the barrel. Advantageously, the variable diameter segments 10, 12 of the barrel 2 allow the plunger 7 to move within the barrel 2 without impeding the movement of the plunger 2 or increasing the glide force necessary to advance the plunger 2 through the barrel 2.

In one example, the negative pressure force is between 0.01 N and 50 N. In another example, the negative pressure force is between 0.05 N and 35 N. In yet another example, the negative pressure force is between 0.1 N and 20 N, or between 0.25 N and 10 N. In some embodiments, the negative pressure force is about 0.01, 0.05, 0.10, 0.25, 0.50, 0.75, 1, 2, 3, 4, 5, 6, 7, 8, 9, 10, 15, 20, 25, 30, 35, 40, 45, 50 N, or more or less, or ranges including any two of the foregoing values.

The particular geometry and dimensions of some embodiments of injectors, such as, for example, including an enlarged inner diameter distal segment 10 with an angled transition zone 14 may create a change in resistive force to plunger 7 axial movement, for example a decrease or reduction in resistive force with plunger 7 distal movement. Such a decrease or reduction in resistive force at the distal end 4 of a drug reservoir (e.g., a syringe barrel 2) can be abrupt to create an audible and/or tactile click feedback as the plunger passes through the resistance transition zone 14. Such a resistance transition zone 14 at the distal end 4 of a drug reservoir (e.g., a syringe barrel) enhances/facilitates the plunger 7 movement distally at the end of injection, which ensures the delivery of the entire dose of the medication. Thus, the lowering of resistance force at the end of injection ensures consistent delivery of the complete medication dose, which can be particularly important for small-dose medicines, for example, those used in volumes of about 0.1 ml or less, or 0.05 ml or less. This is distinct from other mechanisms that can create an obstruction or increased resistive force that impede the plunger 7 movement at the end of injection, which may cause incomplete injection.

In one example, the reduction in resistive force is between 0.01 N and 50 N. In another example, the reduction in resistive force is between 0.05 N and 35 N. In yet another example, the reduction in resistive force is between 0.1 N and 20 N, or between 0.25 N and 10 N. In some embodiments, the reduction in resistive force (e.g., compared to injectors that are otherwise the same or substantially similar without an enlarged inner diameter distal segment 10 with an angled transition zone 14) is about 0.01, 0.05, 0.10, 0.25, 0.50, 0.75, 1, 2, 3, 4, 5, 6, 7, 8, 9, 10, 15, 20, 25, 30, 35, 40, 45, 50 N, or more or less, or ranges including any two of the foregoing values.

In some variations, a space or neck region can be added to separate the luer-lock feature from barrel’s 2 or drug reservoir’s distal end 4. Thus, the attachment of a drug conduit 16 comprising a luer-connector such as a hub to the luer-lock structure 52 of the drug reservoir 2 can advantageously not transmit any force to the distal end of the barrel/reservoir 2 cavity and will not change the internal diameter of the barrel/reservoir 2 cavity that might otherwise result in hampering the click or feedback feature.

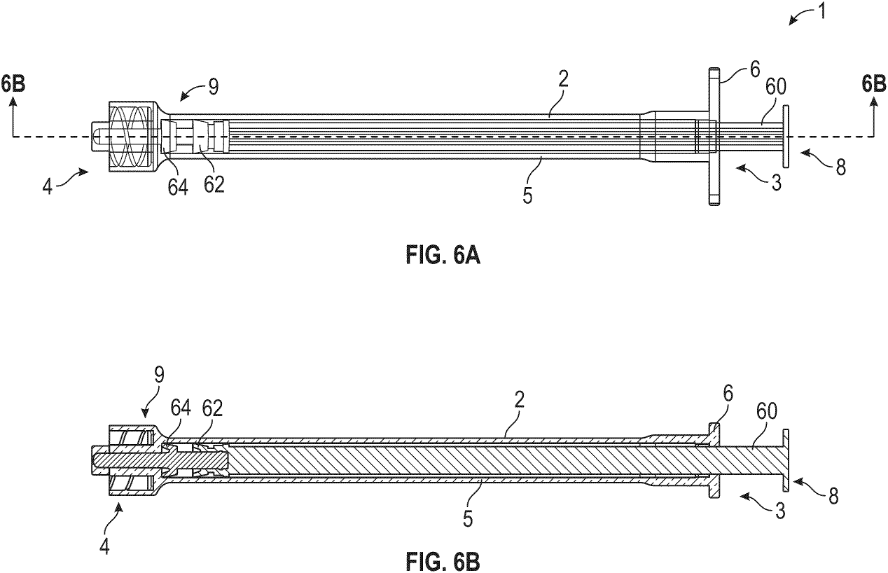

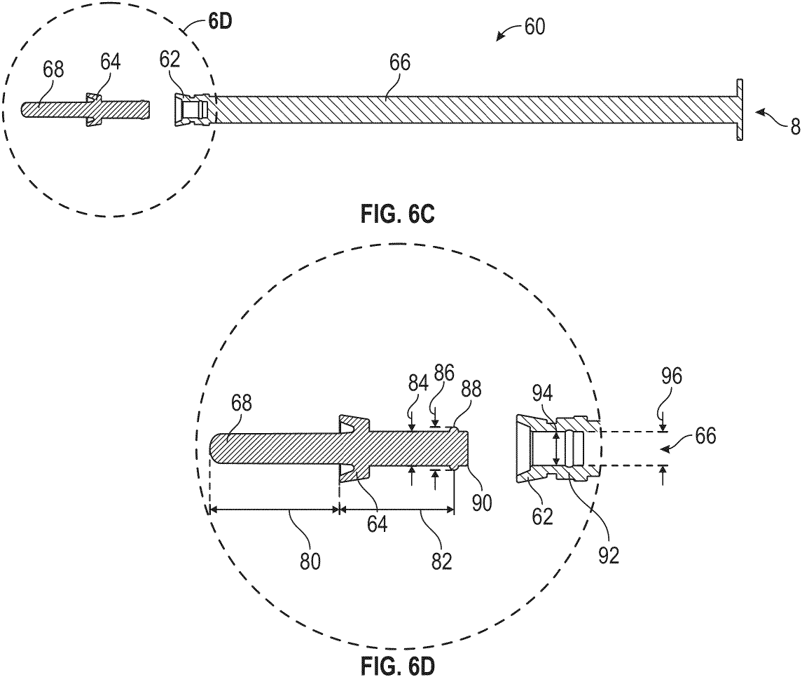

FIG. 5 illustrates an embodiment of a piston syringe 1 having a barrel 2 and a plunger 60 that includes two sealing rings 62, 64 (sometimes referred to as seals or stoppers). The syringe 1 of FIG. 5 is similar to the syringe 1 of FIG. 1, except that the syringe 1 of FIG. 5 includes a cap 70, a two-segment piston 60, and a luer connector integrated into the syringe barrel’s 2 distal end 4. In the illustrated embodiment, the plunger 60 includes two distinct plunger segments 66, 68, each having its own seal, although the plunger 60 may include only one, two, three, four, or more than four plunger segments. The illustrated embodiment includes a single-material plunger 60 having two seals, although in other variation, one, two, three, four, or more than four seals are provided. As a single-material plunger 60, the entire plunger (including the plunger rod and the seal(s)) may be made from the same material. In the illustrated embodiment, the plunger 60 has no rubber or similar material in its stopper, and no lubricant or polymer coating. The multiple-seal plunger 60 ensures there is no leakage past the stoppers and no container closure failure as a result of such leakage. In one variation, the plunger 60 comprises two parts that are molded from the same material. The two-part plunger may be assembled inside a drug reservoir or prior to its insertion into a drug reservoir (e.g., a syringe barrel 2). A multi-seal plunger avoids the potential for the failure of container closure, for example, if the syringe 1 is provided as a drug-prefilled syringe. In addition, a multi-seal plunger ensures proper container closer and prevents leakage, even if one seal fails. For example, with prolonged storage or transport, some syringes, such as a drug-prefilled syringe, could have one seal fail. A second (or additional) seal can assure that no fluid within the syringe leaks outside of the syringe.

The syringe also includes a plunger seal 70 (sometimes referred to as a cap or plunger cap) that covers the proximal end 8 of the plunger 7, 60, and effectively seals the entrance into the barrel 2 of the drug reservoir of the piston syringe 1 to prevents bacterial entry or ingrowth into the barrel 2 even with a prolonged storage of the drug reservoir 1 (e.g., for 30 days, 1 year or 2 years). The cap-seal 70 is long enough to completely enclose the plunger rod/handle 72 when a certain drug volume is present inside the drug reservoir (e.g., 0.05 ml, 0.1 ml, 0.15 ml or more). The cap-seal 70 is attached to the drug reservoir 1 by a thread that provides a secure connection and ensures a water-tight closure. The cap 70 may instead have a slip-on or press-fit connection that also provides a water-tight seal to assure proper container closure. Other coupling mechanisms between the cap 70 and barrel 2 (e.g., slip-on, twisting, clipping, compression fitting, O-ring coupling, etc.) may be provided, as well.

A material such as polypropylene, polyethylene, or any derivative or modification thereof cross-linked with oleic acid lubricant/slip agent may be provided to reduce oleic acid migration into the liquid contents of the drug reservoir (e.g., syringe barrel 2). Crosslinking includes any chemical or physical cross-linking method known to those skilled in the art. For example, crosslinking can be formed by a chemical reaction that is initiated by heat, pressure, change in pH, or irradiation. In one example, the crosslinking method comprises the use of UV light or radiation, infra-red light or radiation, gaseous substance or chemical, a certain temperature, or a combination of these methods.

FIG. 7A illustrates an embodiment of an injection device 1 having a multi-segment plunger 60 and multiple seals 62, 64. FIG. 7B is a cross section of the injection device 1 of FIG. 7A taken along line B-B. FIG. 7C is an exploded view of the multi-segment plunger 60 of FIGS. 7A and 7B. FIG. 7D is a detail view of the distal end of the multi-segment plunger 60 of FIG. 7C taken along line A-A.

The injection device 1 includes a piston 1 having a barrel. The barrel 2 extends from an open proximal end 3 to an open distal end 4 and has a sidewall 5 therebetween. Wing portions 6 extend from the proximal end 3 of the barrel 2. A multi-segment plunger 60 extends through the inner volume of the barrel 2 from the plunger 60 distal end 8 to its proximal end 9. The plunger 60 includes a proximal segment 66 and a distal segment 68. The proximal segment 66 includes a proximal seal 62 to fluidically seal against the inside diameter of the barrel 2. Similarly, the distal segment 68 includes a distal seal 64 to fluidically seal against the inside diameter of the barrel 2.

The distal segment 68 extends a length 80 from the distal seal 64 to the tip or distal end of the distal segment 68. The distal segment 68 extends a length 82 from a bump 88 located near the proximal end 90 of the distal segment 68 to its tip. The bump 88 allows the distal segment 68 to engage and lock into a corresponding notch or groove 96 at the distal end of the proximal segment 66. The diameter 84 of the distal segment 68 includes an enlarged portion 86 that allows a press fit engagement and locking when coupling the distal segment 68 to the proximal segment 66.

The proximal segment 66 includes a coupling region 92 configured to receive and lock onto the proximal end 90 of the distal segment 68. The coupling region 92 includes a cavity 94 sized to receive the outside diameter of the proximal portion of the distal segment when inserted therein.

The injector 1 can be used to inject directly into a patient, into a conduit, or into another medical device. When used directly into a patient, the injection can be intravenous, subcutaneous, intramuscular, intradermal, intraocular, and the like.

The reservoir (e.g., the internal volume of the barrel 2) can be generally contained within the housing or barrel 2 and may be configured in any suitable manner. Some embodiments of reservoirs are configured to deliver a therapeutic agent to an anatomical target region. One non-limiting example of a target region is an intraocular space. The reservoir may hold any suitable drug or formulation, or combination of drugs or formulations, including those disclosed herein. In some embodiments, the therapeutic agent can be delivered to the intraocular space, e.g., the intravitreal space. In some embodiments, the therapeutic agent can be delivered through the outer surface of the skin. In one variation, the reservoir is silicone oil-free (lacks silicone oil or one of its derivatives) and is not internally covered or lubricated with silicone oil, its derivative or a modification thereof, which ensures that silicone oil does not get inside the eye causing floaters or intraocular pressure elevation. In another variation, the reservoir is free of any lubricant or sealant and is not internally covered or lubricated with any lubricating or sealing substance, which ensures that the said lubricating or sealing substance does not get inside the eye causing floaters or intraocular pressure elevation.

In some embodiments, the reservoir includes polypropylene, polyethylene, or a similar polymeric material, as well as any combinations or derivatives thereof. In some embodiments, the reservoir material contains a slip agent. The slip agent may be mixed into the material of the drug reservoir. In some variations, the slip agent is oleic acid, oleic acid amide, another oleic acid derivative, or a similar lubricious material. In some variations, the amount of the slip agent, such as oleic acid amide, is less than 0.6% w/w of the reservoir’s material.

In some variations, the reservoir includes one or more materials that contains a cyclic olefin series resin, a cyclic olefin ethylene copolymer including commercially available products such as Zeonex® cyclo olefin polymer (ZEON Corporation, Tokyo, Japan) or Crystal Zenith® olefinic polymer (Dalkyo Seiko, Ltd., Tokyo, Japan) and APEL® cyclo olefin copolymer (COC) (Mitsui Chemicals, Inc., Tokyo, Japan), a cyclic olefin ethylene copolymer, a polyethylene terephthalate series resin, a polystyrene resin, a polybutylene terephthalate resin, and combinations thereof. In some embodiments, it may be beneficial to use a cyclic olefin series resin and a cyclic olefin ethylene copolymer that have a high transparency, a high heat resistance, and minimal to no chemical interaction with a pharmacological product such as a protein, a protein fragment, a polypeptide, or a chimeric molecule including an antibody, a receptor or a binding protein.

Non-limiting examples of agents may be selected from classes such as anti-inflammatories (e.g., steroidal and non-steroidal), anti-infectives (e.g., antibiotics, antifungals, antiparasitics, antivirals, and antiseptics), cholinergic antagonists and agonists, adrenergic antagonists and agonists, anti-glaucoma agents, neuroprotection agents, agents for cataract prevention or treatment, anti-oxidants, antihistamines, anti-platelet agents, anticoagulants, antithrombotics, anti-scarring agents, anti-proliferatives, anti-tumor agents, complement inhibitors, vitamins (e.g., vitamin B and derivatives thereof, vitamin A, depaxapenthenol, and retinoic acid), growth factors, agents to inhibit growth factors, gene therapy vectors, chemotherapy agents, protein kinase inhibitors, tyrosine kinase inhibitors, PEGF (pigment epithelial growth factor), small interfering RNAs, their analogs, derivatives, conjugates, and modifications thereof, and combinations thereof.

Exemplary complement inhibitors include, but are not limited to, antibodies or blocking peptides that inhibit at least one complement protein or fraction (e.g., anti-C5 agents, including antibodies such as anti-C5a and anti-C5b agents, and ARC1905; anti-C3 agents and antibodies, such as anti-C3 and anti-C3b, and other complement inhibitors, complement fraction inhibitors, or combinations thereof.

Particular agent classes that may be useful include without limitation, anti-neovascularization agents, anti-VEGF agents, anti-platelet derived growth factor agents, anti-placenta derived growth factor agents, anti-pigment epithelium derived growth factor agents, anti-PDGF pathway blocking agents (e.g., a PDGF-beta pathway blocking agent such as anti-PDGF-beta aptamers (e.g., Fovista.TM. anti-PDGF therapy), antibodies, blocking peptides or blocking small molecules), anti-PDGF-beta receptor agents (e.g., a PDGFR-beta blocking agent such as an aptamer, antibody, blocking peptide, or a blocking small molecule), anti-vascular permeability agents, protein kinase C inhibitors, EGF inhibitors, tyrosine kinase inhibitors, steroidal anti-inflammatories, nonsteroidal anti-inflammatories, anti-infectives, anti-allergens, cholinergic antagonists and agonists, adrenergic antagonists and agonists, anti-glaucoma agents, neuroprotection agents, agents for cataract prevention or treatment, anti-proliferatives, anti-tumor agents, complement inhibitors, vitamins, growth factors, agents to inhibit growth factors, gene therapy vectors, chemotherapy agents, protein kinase inhibitors, small interfering RNAs, aptamers, antibodies or antibody fragments, growth factor receptors and receptor fragments, analogs, derivatives, and modifications thereof, and combinations thereof. Further exemplary agents include an anti-complement fraction agent (e.g., an anti-C5 agent, anti-C5a agent, or anti-C3 agent) and aptamers, antibodies, and binding peptides thereof, and combinations thereof. In one variation, a combination of an anti-VEGF agent and an anti-PDGF agent is used.

Non-limiting, specific examples of drugs that may be used alone or as part of a combination drug therapy include Lucentis® (ranibizumab), Avastin® (bevacizumab), Fovista® (anti-PDGF therapy), E10030 aptamer, Macugen® (pegaptanib), anti-complement agents as described above, steroids, e.g., dexamethasone, dexamethasone sodium phosphate, triamcinolone, triamcinolone acetonide, and fluocinolone, taxol-like drugs, integrin or anti-integrin agents, vascular endothelial growth factor (VEGF) trap (aflibercept) (VEGF receptor fragments or analogs), anecortave acetate (Retaane), enzymes, proteases, hyaluronidase, plasmin, ocriplasmin, and limus family compounds, and combinations thereof. Non-limiting examples of members of the limus family of compounds include sirolimus (rapamycin) and its water soluble analog SDZ-RAD, tacrolimus, everolimus, pimecrolimus, and zotarolimus, as well as analogs, derivatives, conjugates, salts, and modifications thereof, and combinations thereof. In some instances, it may be beneficial to employ a combination of agents. For example, it may be beneficial to combine two or more of the following for therapy: Lucentis® (ranibizumab), Fovista® (anti-PDGF therapy), Eylea® (aflibercept), an anti-PDGF agent, Macugen®, Jetrea®, a thrombolytic agent, and a steroid. In some instances, it may be beneficial to combine Lucentis® (ranibizumab) and Fovista® (or another anti-PDGF agent) combination, or Eylea® (aflibercept) and Fovista® (or another anti-PDGF agent).

Topical anesthetic agents may also be included in the reservoirs. For example, lidocaine, proparacaine, prilocaine, tetracaine, betacaine, benzocaine, ELA-Max®, EMLA® (eutectic mixture of local anesthetics), and combinations thereof may be used.

In some embodiments, features of injectors as disclosed herein can be utilized with pre-filled syringes (PFS) of one, two, or more agents as disclosed, for example, herein, including but not limited to ranibizumab-PFS, aflibercept-PFS, conbercept-PFS, razumab-PFS, bavacizumab-PFS, or biosimilars of the foregoing.

Some variations of the injection devices described herein include a filter that filters the contents of the reservoir as it is delivered into the desired location, such as, for example, the eye. For example, the filter may be used to remove infectious agents and enhance sterility of a therapeutic agent formulation before injection. Thus, inclusion of a filter into the device may be useful because, for example, the eye is an immune-privileged site, and introduction of even a small quantity of pathogens such as bacteria may cause sight-threatening intraocular infection (endophthalmitis). The filter may also be used to remove impurities, e.g., silicone droplets, from a therapeutic agent formulation prior to injection into the eye. This may be useful for intraocular drugs because a small impurity injected into a subject’s eye may result in the subject seeing it as floater(s) that may be intractable, which significantly worsens the quality of vision.

In some embodiments, an injector 1 can include an enhanced-sterility syringe loading kit comprising a needle and terminal sterilization mechanism that ensure sterility of syringe drug loading. The needle could be a shielded loading needle in some embodiments. In some embodiments, a needle, such as a sliding-cap therapeutic agent loading needle, for example, can be removably attached to and packaged together with a reservoir such as a piston syringe (e.g., luer-lock or luer-slip) inside a sterile package, such as a tray or blister package.

In some embodiments, a needle can include sterilizing filter integrated in its hub (e.g., 0.2, 0.5, or other micron pore size). The filter can include, for example, materials as disclosed elsewhere herein.

In one variation, the filter pore size is between about 0.1 µm and about 10 µm, between about 0.2 µm to about 5.0 µm, or between about 0.2 µm and about 1.0 µm to facilitate filtration of bacterial pathogens, particulate matter or impurities such as silicone droplets from the outgoing drug being injected intraocularly. Thickness of the said may range from between about 50 µm to about 250 µm, or from between about 10 µm to about 10,000 µm.

The filter may be made from any suitable non-reactive material, such as a low protein-binding material. Exemplary filter materials include without limitation, thermoplastic fluoropolymers such as PVDF (polyvinylidene fluoride); thermoplastic polymers such as polyethylene and polypropylene; mixed cellulose esters; nylons; polyesters; nitrocelluloses; acrylic polymers such as Versapor® acrylic copolymer (Pall, Inc.); polyethersulfones such as found in Supor® (Pall, Inc.) filters; a combination, a mixture, or a blend thereof. The filter may be hydrophilic in some cases to enable filtration-sterilization of pathogens such as bacteria from a therapeutic agent solution. In one example, the filter comprises a pathogen-filtering membrane that has a low protein-binging capacity to avoid binding and retention of protein-based pharmaceuticals.

The filter may be integrated with the device housing 2, the reservoir, the conduit, or any part of the device 1. In one variation, the filter is press-fit into a device lumen, for example into the lumen of a male-type luer, or a female-type hub, such as a drug conduit hub. In one variation, the filter is internal to the device 1. For example, the filter is configured to be inside the drug reservoir, or inside the conduit, or at the junction between reservoir and conduit. In another variation, filter is detachable or removable from the device 1. In one variation, the filter is located within the reservoir at its distal end, e.g., within the luer of a syringe. In another variation, the filter is located at the proximal end of the lumen of the conduit. The filter may also be placed at any location within and along the lumen of the drug delivery conduit, e.g., at its proximal end, in the middle, or at the distal end of the conduit.

In one example, the filter is integrated with the drug-loading conduit or device utilized to load a drug into the intraocular drug delivery devices described herein. For example, the filter is located inside the drug-loading conduit, or at or near the internal opening of the lumen of the drug-loading conduit. The filter may also be placed at any location within and along the lumen of the drug-loading conduit, e.g., at its proximal end, in the middle, or at the distal end of the conduit. For example, integrating a sterilizing filter within the drug-loading conduit may prevent microbial pathogens from room air from being introduced into the drug during the loading procedure.

In addition to removing infectious agents and/or impurities from the reservoir contents, the filter may function as a jet control mechanism that controls the force and limits the travel distance of the injected fluid as it exits the device and enters the eye. Other configurations of the jet control mechanism are also contemplated. The jet control mechanism may be generally configured to limit the maximum travel distance of the injected fluid to between about 5 mm and about 25 mm, between about 5 mm to about 20 mm, between about 5 mm and about 15 mm, or between about 5 mm to about 10 mm. In some instances, the maximum travel distance may be limited to less than about 25 mm, less than about 15 mm, less than about 10 mm, or less than about 5 mm. When a filter serves as the jet control mechanism, the pore size may range from about 0.05 µm to about 10 µm, from about 0.1 µm to about 5 µm, or from about 0.2 µm to about 1 µm. Such a filter may also be placed within any portion of the device, e.g., near the device conduit.

The jet control mechanism may also include a fluid displacement control mechanism. The fluid displacement control mechanism may include a plunger 7 rate control mechanism such as a mechanical interference, resistance component, or pneumatic control component that is configured to control the rate of plunger 7 advancement within the reservoir of the device 1. The jet control mechanism may be beneficial because it improves the safety of intraocular drug injections, e.g., by minimizing the risk of serious adverse effects such as retinal detachment or other types of damage to intraocular structures by a forceful jet of fluid inside the eye.

It may also be advantageous to remove air from the reservoir contents before it is injected into the eye since the presence of intraocular air can result in unpleasant visual disturbances (“floaters”). The removal of air from a viscous composition, e.g., a viscous drug solution such as Lucentis® (ranibizumab injection), may be particularly beneficial. Thus, in some variations, the devices described herein may also include an air control mechanism for removing the amount of air introduced into the eye during intraocular drug administration. The air removal mechanism may be configured as a filter, a plurality of filters, a valve, a reservoir, or a combination of any of the foregoing. The air removal mechanism may be placed within any portion of the device 1, e.g., near the device conduit.

Some variations of the air control mechanism may include a hydrophobic filter or porous hydrophobic membrane that allows air through while retaining an aqueous drug solution. Exemplary materials that may be employed in the hydrophobic filters include without limitation, polytetrafluoroethylene (PTFE), Supor® Membrane (Pall Corporation, Ann Arbor, Mich.), Versapor® Membrane (Pall Corporation, Ann Arbor, Mich.), and other porous filter materials that have been coated or treated with a hydrophobic membrane such as Repel® Acrylic Copolymer Membrane (Pall Corporation, Ann Arbor, Mich.). The pore size of the air removal filters may range from about 0.05 µm to about 50 µm, from about 0.1 µm to about 10 µm, or from about 0.2 µm to about 5 µm.

Some variations of the air control mechanism may include a (e.g., proximal) extension of the drug conduit and its internal opening (or its holder or internal opening of the hub of the drug conduit) into the internal cavity of a syringe luer or drug reservoir (e.g., beyond the internal surface plane of the hub). Such proximal extension of the drug conduit and its internal opening beyond the internal surface of its hub may provide an internal hub-luer seal (e.g., between the female-type hub, such as a disposable needle hub, and male-type luer, such as a syringe luer), where the seal prevents air and/or fluid in the drug solution or suspension from entering the cavity of the needle hub, but rather enables the air and/or fluid to pass directly from a drug reservoir into the drug conduit. For example, the said internal hub-luer seal may be generated by fit interference between the external surface of the proximal extension of the drug conduit or its internal hub continuation and the internal surface of the male-type luer of a drug reservoir, such as a syringe luer.

Various other modifications, adaptations, and alternative designs are of course possible in light of the above teachings. Therefore, it should be understood at this time that within the scope of the appended claims the invention may be practiced otherwise than as specifically described herein. It is contemplated that various combinations or subcombinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. For example, actions such as “applying pressure on a plunger to create an audible and/or tactile click feedback” includes “instructing the applying of pressure on a plunger to create an audible and/or tactile click feedback.” The ranges disclosed herein also encompass any and all overlap, sub-ranges, and combinations thereof. Language such as “up to,” “at least,” “greater than,” “less than,” “between,” and the like includes the number recited. Numbers preceded by a term such as “approximately”, “about”, and “substantially” as used herein include the recited numbers (e.g., about 10% = 10%), and also represent an amount close to the stated amount that still performs a desired function or achieves a desired result. For example, the terms “approximately”, “about”, and “substantially” may refer to an amount that is within less than 10% of, within less than 5% of, within less than 1% of, within less than 0.1% of, and within less than 0.01% of the stated amount.

1. An injection device, comprising:
a barrel comprising an open proximal end, an open distal end, and a sidewall therebetween, the barrel configured to house a volume of therapeutic agent;
a plunger comprising a proximal end, a distal end, and an elongate body; and
a conduit or connector operably attached to the open distal end of the barrel, wherein:
the distal end of the barrel comprises a variable inner diameter segment configured such that movement of the plunger to the variable inner diameter segment of the distal end of the barrel creates an audible and/or tactile feedback sensation to a user;
the variable inner diameter segment comprises a first segment and a second segment distal to the first segment, the second segment comprising an inner diameter that is greater than an inner diameter of the first segment; and
the variable inner diameter segment comprises a transition zone between the first segment and the second segment.

2. The device of claim 1, wherein the device comprises a piston syringe.

3. The device of claim 1, wherein an inner diameter of the barrel proximal to the variable inner diameter segment is substantially constant.

4. (canceled)

5. The device of claim 1, wherein the inner diameter of the second segment is between about 0.1 mm and about 0.5 mm greater than the inner diameter of the first segment.

6. The device of claim 5, wherein the inner diameter of the second segment is about 0.3 mm greater than the inner diameter of the first segment.

7. The device of claim 1, wherein the second segment has an axial length of between about 0.05 mm and about 0.20 mm.

8. (canceled)

9. The device of claim 1, wherein the transition zone comprises an axial length that is less than, and within about 0.05 mm of the length of the second segment.

10. The device of claim 1, wherein the transition zone comprises an axial length that is less than, and within about 0.03 mm of the length of the second segment.

11. The device of claim 1, wherein the transition zone is slanted at an angle Z with respect to a long axis of the sidewall of the barrel, wherein the angle Z is less than about 90 degrees.

12. The device of claim 1, wherein the transition zone is slanted at an angle Z with respect to a long axis of the sidewall of the barrel, wherein the angle Z is less than about 45 degrees.

13. The device of claim 1, wherein the transition zone is slanted at an angle Z with respect to a long axis of the sidewall of the barrel, wherein the angle Z is greater than about 45 degrees.

14. The device of claim 1, wherein the movement of the plunger to the variable inner diameter segment of the distal end of the barrel creates negative pressure within the barrel sufficient to fully inject the entire volume of the barrel.

15. The device of claim 1, wherein the distal end of the plunger comprises a variable inner diameter segment that is substantially the same length and inner diameter as the variable inner diameter segment of the distal end of the barrel.

16. The device of claim 1, wherein the variable inner diameter segment at the distal end of the barrel is less than about 10% of the total axial length of the barrel.

17. (canceled)

18. (canceled)

19. (canceled)

20. A method of injecting a therapeutic agent, comprising:
providing an injector comprising:
a barrel comprising an open proximal end, an open distal end, and a sidewall therebetween, the barrel configured to house a volume of therapeutic agent;
a plunger comprising a proximal end, a distal end, and an elongate body; and
a conduit or connector operably attached to the open distal end of the barrel, wherein the distal end of the barrel comprises a variable inner diameter segment; and
actuating the plunger to purge the volume of therapeutic agent out of the barrel, such that movement of the plunger to the variable inner diameter segment of the distal end of the barrel creates an audible and/or tactile feedback sensation to a user; wherein:
the variable inner diameter segment comprises a first segment and a second segment distal to the first segment, the second segment comprising an inner diameter that is greater than an inner diameter of the first segment;
the inner diameter of the second segment is between about 0.1 mm and about 0.5 mm greater than the inner diameter of the first segment; and
the variable inner diameter segment comprises a transition zone between the first segment and the second segment.

21. The method of claim 20, wherein movement of the plunger to the variable inner diameter segment of the distal end of the barrel creates negative pressure within the barrel to accentuate an audible and/or tactile feedback sensation to the user.

22. (canceled)

23. The method of claim 20, wherein an inner diameter of the barrel proximal to the variable inner diameter segment is substantially constant.

24. (canceled)

25. (canceled)

26. The method of claim 20, wherein the inner diameter of the second segment is about 0.3 mm greater than the inner diameter of the first segment.

27. The method of claim 20, wherein the second segment has an axial length of between about 0.05 mm and about 0.20 mm.

28. (canceled)

29. The method of claim 20, wherein the transition zone comprises an axial length that is less than, and within about 0.05 mm of the length of the second segment.

30. The method of claim 20, wherein the transition zone comprises an axial length that is less than, and within about 0.03 mm of the length of the second segment.

31. The method of claim 15, wherein the transition zone is slanted at an angle Z with respect to a long axis of the sidewall of the barrel, wherein the angle Z is less than about 90 degrees.

32. The method of claim 20, wherein the transition zone is slanted at an angle Z with respect to a long axis of the sidewall of the barrel, wherein the angle Z is less than about 45 degrees.

33. The method of claim 20, wherein the transition zone is slanted at an angle Z with respect to a long axis of the sidewall of the barrel, wherein the angle Z is greater than about 45 degrees.

34. The method of claim 20, wherein the therapeutic agent comprises an anti-VEGF agent.

35. (canceled)

36. (canceled)

37. (canceled)

38. The method of claim 20, wherein the therapeutic agent is pre-filled within the barrel.

39. An injection device, comprising:
a barrel comprising an open proximal end, an open distal end, and a sidewall therebetween, the barrel configured to house a volume of therapeutic agent and defining a therapeutic agent reservoir;
a plunger comprising a proximal end, a distal end, and an elongate body; and
a conduit or connector operably attached to the open distal end of the barrel,
wherein the distal end of the barrel comprises a variable inner diameter segment configured such that movement of the plunger to the variable inner diameter segment of the distal end of the barrel creates an audible and/or tactile feedback sensation to a user while passing through the variable inner diameter segment,
wherein the variable inner diameter segment comprises a first, narrower proximal part, a second, wider distal part, and a third angled transition zone part between the first part and the second part.

40. The injection device of claim 39, wherein the conduit comprises a removable or non-removable conduit.

41. (canceled)

42. (canceled)

43. The injection device of claim 39, wherein the second part is adjacent to a distal wall of the therapeutic agent reservoir.

44. The injection device of claim 39, wherein the distal end of the plunger is sized and configured to pass through the first part, second part, and third part of the variable inner diameter segment without losing an air and/or fluid tight seal.

45-50. (canceled)
